(19) United States
(12) Patent Application Publication
(10) Pub. No.: US 2021/0128200 A1
(43) Pub. Date: May 6, 2021

(54) OCULAR DEBRIS REMOVAL DEVICE

(71) Applicant: Steven Easley, O'Fallon, MO (US)

(72) Inventor: Steven Easley, O'Fallon, MO (US)

(21) Appl. No.: 16/669,659

(22) Filed: Oct. 31, 2019

(51) Int. Cl. A61B 17/52; A61F 9/007

(52) U.S. Cl. CPC A61B 17/52; A61F 9/00709

(57) ABSTRACT

An ocular debris removal device for removing magnetic debris from the eye includes a handle with a first end, a second end, and a body extending between the first and second ends. A magnet is attached to the second end of the handle and extends away therefrom. The magnet has an attached end and a distal end with respect to the handle. The magnet has a perimeter surface between the attached and distal ends wherein the perimeter surface bounds a center longitudinal axis of the magnet. The center longitudinal axis of the magnet is co-linear with a center longitudinal axis of the handle extending through the first and second ends.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to ocular debris removal devices and more particularly pertains to a new ocular debris removal device for removing magnetic debris from the eye.

### (2) Description of Related Art Including Information Disclosed Under 37 CFR 1.97 and 1.98

The prior art relates to ocular debris removal devices used for removing metallic and non-metallic particles from a person's eye.

### BRIEF SUMMARY OF THE INVENTION

An embodiment of the disclosure meets the needs presented above by generally comprising a handle with a first end, a second end, and a body extending between the first and second ends. A magnet is attached to the second end of the handle and extends away therefrom. The magnet has an attached end and a distal end with respect to the handle. The magnet has a perimeter surface between the attached and distal ends wherein the perimeter surface bounds a center longitudinal axis of the magnet. The center longitudinal axis of the magnet is co-linear with a center longitudinal axis of the handle wherein the center longitudinal axis of the handle extends through the first and second ends.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an ocular debris removal device according to an embodiment of the disclosure.

FIG. 2 is a front view of an embodiment of the disclosure illustrating the device from the distal end of the magnet.

FIG. 3 is a side view of an embodiment of the disclosure illustrating the side profile of the device.

FIG. 4 is a section view of an embodiment of the disclosure illustrating the interior features of the device when bisected lengthwise about the line illustrated in FIG. 2.

FIG. 5 is an instructional illustration of how to operate an embodiment of the disclosure.

### DETAILED DESCRIPTION OF THE INVENTION

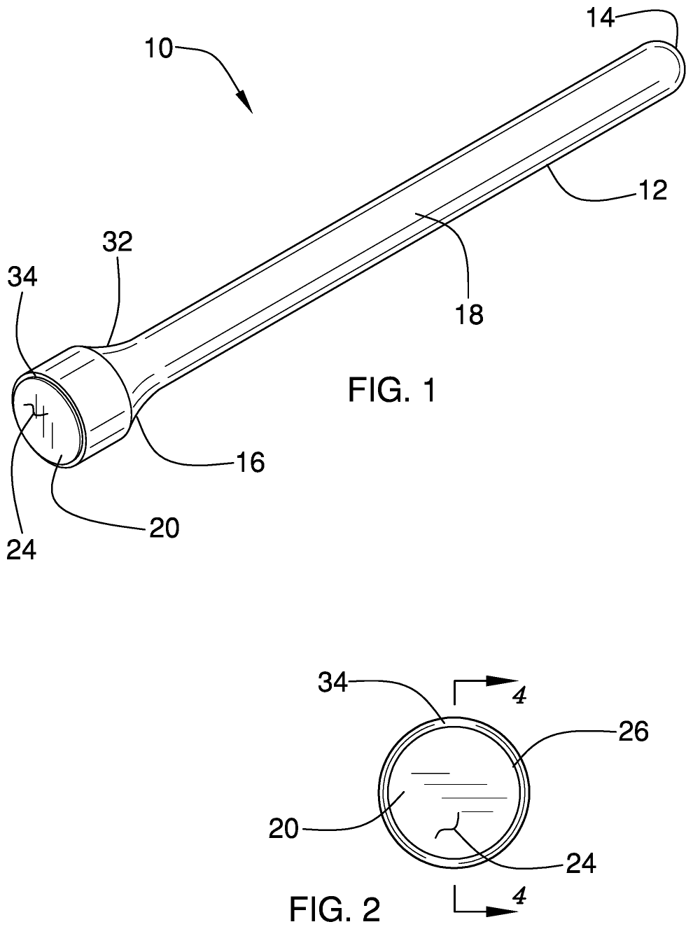

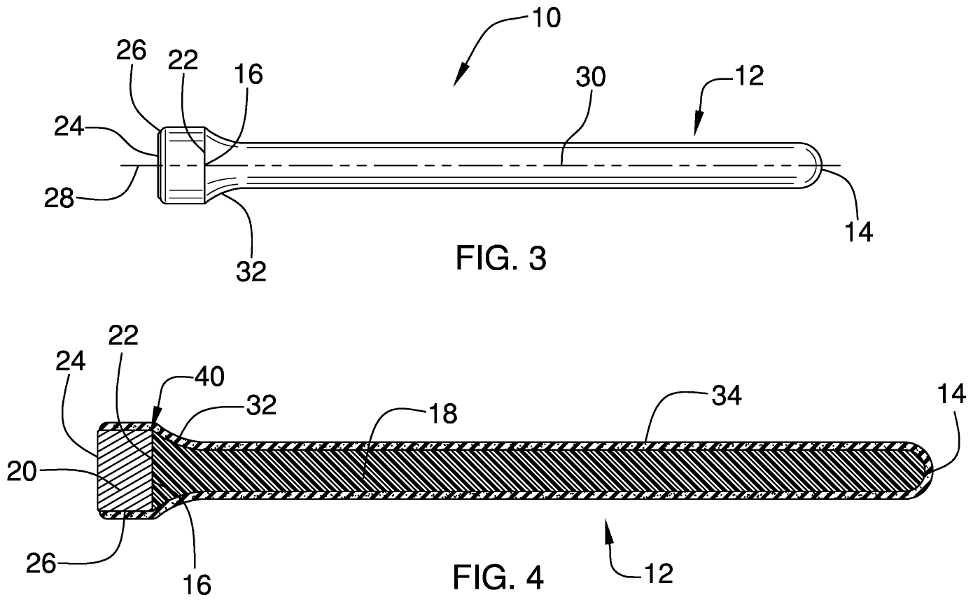

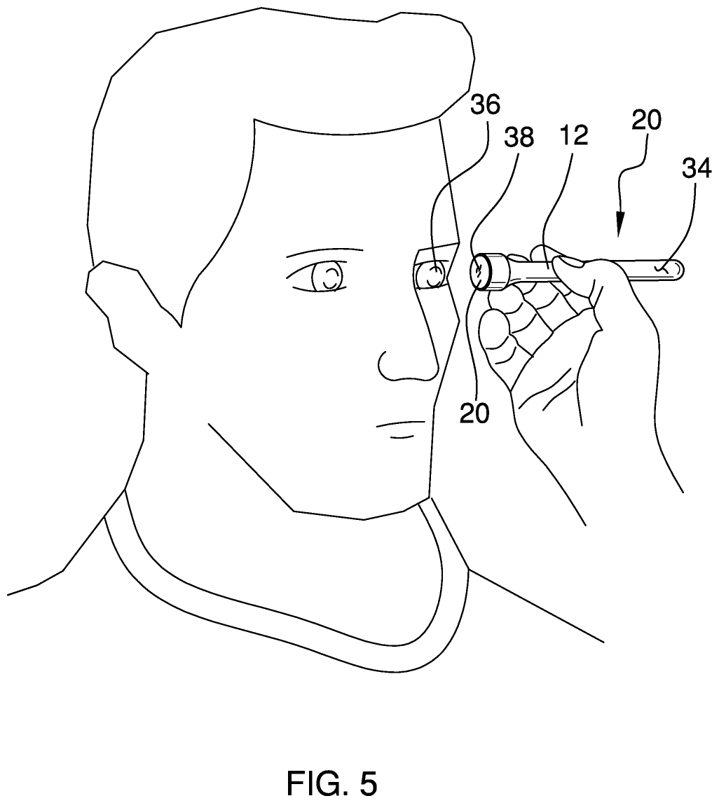

With reference now to the drawings, and in particular to FIGS. 1 through 5 thereof, a new ocular debris removal device embodying the principles and concepts of an embodiment of the disclosure and generally designated by the reference numeral 10 will be described.

As best illustrated in FIGS. 1 through 5, the ocular debris removal device 10 generally comprises a handle 12 with a first end 14, a second end 16, and a body 18 extending between the first 14 and second 16 ends. The body 18 has a cylindrical shape and has a length between 6.00 and 7.00 inches. The first end 14 may be rounded to form a semi-spherical geometry.

A magnet 20 is attached to the second end 16 of the handle 12 and extends away therefrom. The magnet 20 has an attached end 22 and a distal end 24 with respect to the handle 12. The magnet 20 has a perimeter surface 26 between the attached 22 and distal 24 ends. The perimeter surface 26 forms a cylindrical shape and has a diameter between 0.50 and 1.00 inches. The perimeter surface 26 bounds a center longitudinal axis 28 of the magnet 20. The center longitudinal axis 28 of the magnet 20 is co-linear with a center longitudinal axis 30 of the handle 12. The center longitudinal axis 30 of the handle 12 extends through the first 14 and second 16 ends. The magnet 20 may be of any magnetic or magnetizable material.

The handle 12 increases in diameter adjacent to the magnet 20 to define a shoulder 32 at a juncture 40 of the magnet 20 and the second end 16. The first end 14 has a diameter between 0.25 and 0.75 inches. The second end 16 has a diameter between 0.50 and 1.00 inches.

A coating 34 is configured to aid in holding the handle 12 and is positioned on and completely covers the first end 14 and the body 18 of the handle 12. The coating 34 extends from the handle 12 over at least a portion of the perimeter surface 26 of the magnet 20. The distal end 24 is free of the coating 34. The coating 34 comprises an elastomeric material.

In use, the ocular debris removal device 10 is grasped by the handle 12. The distal end 24 of the magnet 20 is positioned near, but not in contact with, the affected eye 36. The magnet 20 pulls the magnetic debris 38 directly away from the affected eye 36 and deposits the magnetic debris 38 onto the distal end 24. The debris 38 is then removed from the distal end 24.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A debris removal magnet assembly configured to assist in removing magnetic debris from an affected eye, said assembly comprising:
a handle having a first end, a second end, and a body extending between said first and second ends; and
a magnet being attached to said second end of said handle, said magnet extending away from said second end of said handle, said magnet having an attached end and a distal end with respect to said handle, said magnet having a perimeter surface between said attached and distal ends wherein said perimeter surface bounds a center longitudinal axis of said magnet, said center longitudinal axis of said magnet being co-linear with a center longitudinal axis of said handle, said center longitudinal axis of said handle extends through said first and second ends of said handle.

2. The debris removal magnet assembly according to claim 1, wherein said body has a cylindrical shape.

3. The debris removal magnet assembly according to claim 1, wherein said body measures between 6.00 and 7.00 inches in length.

4. The debris removal magnet assembly according to claim 2, wherein said body measures between 6.00 and 7.00 inches in length.

5. The debris removal magnet assembly according to claim 1, wherein said perimeter surface forms a cylindrical shape.

6. The debris removal magnet assembly according to claim 5, wherein said perimeter surface has a diameter between 0.50 and 1.00 inches.

7. The debris removal magnet assembly according to claim 1, wherein said handle increases in diameter adjacent to said magnet to define a shoulder at a juncture of said magnet and said second end.

8. The debris removal magnet assembly according to claim 7, wherein said first end has a diameter between 0.25 and 0.75 inches, and said second end has a diameter between 0.50 and 1.00 inches.

9. The debris removal magnet assembly according to claim 1, further comprising a coating being positioned on and completely covering said first end and said body of said handle.

10. The debris removal magnet assembly according to claim 9, wherein said coating extends from said handle over at least a portion of said perimeter surface of said magnet, said distal end being free of said coating.

11. The debris removal magnet assembly according to claim 9, wherein said coating comprises an elastomeric material.

12. The debris removal magnet assembly according to claim 10, wherein said coating comprises an elastomeric material

13. A debris removal magnet assembly configured to assist in removing magnetic debris from an affected eye, said assembly comprising:
a handle having a first end, a second end, and a body extending between said first and second ends, said body having a cylindrical shape wherein said body measures between 6.00 and 7.00 inches in length;
a magnet being attached to said second end of said handle and extending away therefrom, said magnet having an attached end and a distal end with respect to said handle, said magnet having a perimeter surface between said attached and distal ends, said perimeter surface forming a cylindrical shape, said perimeter surface having a diameter between 0.50 and 1.00 inches, said perimeter surface bounding a center longitudinal axis of said magnet, said center longitudinal axis of said magnet being co-linear with a center longitudinal axis of said handle extending through said first and second ends;
said handle increasing in diameter adjacent to said magnet to define a shoulder at a juncture of said magnet and said second end wherein said first end has a diameter between 0.25 and 0.75 inches, said second end has a diameter between 0.50 and 1.00 inches; and
a coating being positioned on and completely covering said first end and said body of said handle, said coating extending from said handle over at least a portion of said perimeter surface of said magnet, said distal end being free of said coating, said coating comprising an elastomeric material.

14. A method of removing magnetic debris from an affected eye including the steps of:
holding a debris removal magnet assembly near an affected eye, said debris removal magnet assembly including a handle having a first end, a second end, and a body extending between said first and second ends, said body having a cylindrical shape wherein said body measures between 6.00 and 7.00 inches in length, a magnet being attached to said second end of said handle and extending away therefrom, said magnet having an attached end and a distal end with respect to said handle, said magnet having a perimeter surface between said attached and distal ends, said perimeter surface forming a cylindrical shape, said perimeter surface having a diameter between 0.50 and 1.00 inches, said perimeter surface bounding a center longitudinal axis of said magnet, said center longitudinal axis of said magnet being co-linear with a center longitudinal axis of said handle extending through said first and second ends, said handle increasing in diameter adjacent to said magnet to define a shoulder at a juncture of said magnet and said second end wherein said first end has a diameter between 0.25 and 0.75 inches, said second end has a diameter between 0.50 and 1.00 inches, and a coating being positioned on and completely covering said first end and said body of said handle, said coating extending from said handle over at least a portion of said perimeter surface of said magnet, said distal end being free of said coating, said coating comprising an elastomeric material; and
placing said debris removal magnet assembly near, but not in contact, with the affected eye until magnetic debris positioned on the affected eye dislodges from the affected eye and is and magnetically fixed to said distal end.
